(19) United States
(12) Patent Application Publication
(10) Pub. No.: US 2024/0066311 A1
(43) Pub. Date: Feb. 29, 2024

(54) Hyperthermia Electromagnetic Energy Applicator Housing and Hyperthermia Patient Support System

(71) Applicant: Pyrexar Medical Inc., Salt Lake City, UT (US)

(72) Inventors: Jason Ellsworth, Farmington, UT (US); Paul F. Turner, Bountiful, UT (US)

(21) Appl. No.: 17/985,818

(22) Filed: Nov. 11, 2022

Related U.S. Application Data
(63) Continuation-in-part of application No. 15/190,098, filed on Jun. 22, 2016, now abandoned.

(51) Int. Cl. A61N 5/04; A61N 5/02

(52) U.S. Cl. CPC A61N 5/04; A61N 5/025; A61N 2005/027

(57) ABSTRACT

EMR applicators of an EMR applicator array are provided in an openable applicator housing to allow easy patient entrance to and exit therefrom so that the portion of the patient's body that contains the tissue to be treated can be positioned directly into the applicator housing without the applicator housing having to be moved along the patient's body. This can eliminate or reduce the need for full body supporting structure as part of the applicator housing. The applicator housing provides support for the portion of the body positioned therein, and support pads or pillows, separate from the applicator housing, can replace the need for a full body support as part of the applicator housing. The housing can reduce the size and complication of prior art applicator housings, and can be placed along with the patient, on a patient support surface, such as in a standard MRI system.

### BACKGROUND OF THE INVENTION

Field: The present invention relates generally to systems and apparatus for irradiating patients with electromagnetic radiation, and more specifically to systems having annular-type or sectored arrays of applicators and associated control systems for controlling application of radiation to patients through phased array power steering, wherein the patient is supported within the annular-type or sectored array of applicators wherein the applicators surround a portion of the patient.

State of the Art: Hyperthermia, the generation of artificially elevated body temperatures, has recently been given serious scientific consideration as an alternative cancer treatment. Much research has been conducted into the effectiveness of hyperthermia alone or in combination with other treatment methods. Hyperthermia techniques appear to have the potential for being extremely effective in the treatment of many or most types of human cancers, without the often severely adverse side effects associated with current cancer treatments such as chemotherapy or radiation. Hyperthermia is sometimes called thermal therapy indicating the raising of the temperature of a region of the body.

Hyperthermia is generally provided by temperatures over 40 degrees C. (104 degrees F.). Hyperthermia has historically included temperatures well above 60 degrees C., but in recent years has generally been considered to include temperatures as high as 45 degrees C. (113 degrees F.). Currently treatments using temperatures well above 45 degree C. to directly kill or ablate tissue, such as cancer tissue, is usually referred to as ablation rather than hyperthermia, but the term hyperthermia as used herein can include either type of heating and treatment. At treatment temperatures above the approximate 45 degrees C. (113 degrees F.), thermal damage to most types of normal cells is routinely observed if the time duration exceeds 30 to 60 minutes; thus, great care must be taken not to exceed these temperatures in healthy tissue for a prolonged period of time. Exposure duration at any elevated temperature is an important factor in establishing the extent of thermal damage to healthy tissue. If large or critical regions of the human body are heated into, or above, the 45 degree C. temperature range for even relatively short times, normal tissue injury may be expected to result. With any such heat treatment, the intent is to get as much of the cancerous tissue as possible above the 40 degree C. temperature, without heating the normal tissue surrounding the cancerous tissue to temperatures which will kill or damage the normal tissue. Therefore, it is desirable to be able to selectively heat cancerous tissue in a mass of normal tissue, such as in a human body, to desired increased temperatures without heating the normal tissue.

One way to heat tissue is to apply electromagnetic radiation (EMR) to such tissue. One currently used method for applying electromagnetic radiation (EMR) to selected targets, such as living bodies and biological tissue, and for controlling the position of a region of heating within the target is through phased array power steering. Systems that use phased array power steering provide a plurality of electromagnetic energy applicators positioned around a portion of a living body or tissue mass to be treated wherein the power and phase of the electromagnetic energy radiated by each electromagnetic energy applicator can be controlled to control the size and location of an area of heating within the living body or tissue mass. Generally, the plurality of electromagnetic energy applicators will be positioned to surround the living body or tissue mass to be treated. With such systems, the array of electromagnetic energy applicators are generally provided in a housing wherein the electromagnetic energy applicators form at least one ring within the housing around an opening in the housing adapted to receive the living body or tissue mass therein. When the living body or tissue mass is a human patient, the patient is supported with the portion of the patient containing the tissue to be treated, such as the pelvis, abdomen, or thorax of the patient, within the opening of the housing so that the electromagnetic energy applicators in the housing substantially encircle the portion of the patient containing the tissue to be treated. With such arrangement, the electromagnetic energy applicators are spaced around the patient a distance away from the patient. The housing generally includes an inflatable bolus around the opening which can be filled with a dielectric fluid having an impedance approximately equivalent to an applicator impedance at the frequency of the EMR energy radiation being used in the system to fill the space between the electromagnetic energy applicators and the surface (skin) of the portion of the patient received in the opening. The dielectric fluid will generally be deionized water. Examples of such prior art systems are shown and described in U.S. Pat. Nos. 4,672,980; 5,097,844; 7,565,207; and 8,170,643, all of which are incorporated herein by reference. A commercial system is available as the BSD-2000 system from Pyrexar Medical Inc. in Salt Lake City, Utah.

As indicated, prior art systems, such as the BSD-2000, provide a housing which includes an opening through which the patient is inserted and the portion of the patient having the tissue to be treated is in the opening. With the patient positioned in the opening in the housing, a bolus, included as part of the housing, is filled with deionized water to contact the portion of the patient in the opening in the housing and provide the deionized water between the electromagnetic energy applicators in the housing and the patient's body portion positioned in the opening in the housing. In order to accurately position the patient in the opening in the housing, a fabric sling patient support is provided. FIGS. 1 and 2 show BSD-2000 systems with a patient 10 positioned in opening 12 in applicator housing 14, which includes a cylindrical applicator holder 16 with bolus 18 inflated with deionized water around the patient. As used herein, cylindrical does not mean circular in cross section as the illustrated cylindrical applicator holder 16 is shown as being substantially elliptical in cross section, but merely means that it surrounds the patient. The electromagnetic energy applicators are secured to and spaced around the inside of the cylindrical applicator holder 16, and are inside the bolus 18. The patient 10 is supported on a fabric sling 20 supported along opposite sides by side support tubes 22. The ends of the respective side support tubes 22 are connected by end support tubes 24. Brackets 26 connect side support tubes 22 through end support tubes 24 to vertical corner supports 28. In the embodiment of FIG. 1, the vertical corner supports 28 extend from system base 30. Vertical corner supports 28, and brackets 26 attached thereto, are height adjustable to enable the fabric sling 20 to be height adjustable for adjusting the height and inclination of the sling 20 and patient 10 thereon in the applicator housing 14. The applicator housing 14 is mounted on track 32 for longitudinal movement on system base 30 with respect to sling 20 and patient 10 thereon to align the applicator housing 14 with the portion of the patient's body containing the tissue to be treated. With this arrangement, the applicator housing 14 can be moved along the track 32 to an end of the sling 20 to allow easy access of the patient to or exit of the patient from sling 20. Once the patient is on sling 20, applicator housing 14 is moved along track 32 and sling 20 to the desired position along the patient's body. When positioned as desired along the patient's body, such as around the patient's abdomen as shown in FIG. 1, the height of the patient's body in applicator opening 12 with respect to the cylindrical applicator holder 16 in applicator housing 14 can be adjusted by adjusting corner supports 28. Vertical corner supports 28 are generally controlled and adjusted by hydraulic lifts in system base 30. When in desired position, bolus 18 is filled with deionized water to contact the patient above the sling 20 and to contact the bottom of the sling 20 which serves as the contact of the bolus with the portion of the patient resting on sling 20, the bolus also forming itself around the edges of the sling and the sling side supports 22. This arrangement works satisfactorily for a system such as shown in FIG. 1 where the system performs its treatment standing as shown in FIG. 1. The height and tilt adjustment of the patient in the applicator housing as provided by the fabric sling 20 and adjustable corner supports 28 is important because hyperthermia treatments are usually repeatedly performed at time intervals, such as weekly, for a predetermined plurality of treatments. Each treatment should be the same as the previous treatments which means that the patient has to be in the same position in the applicator housing for each treatment. The adjustability is necessary so the position of the patient for each treatment can be adjusted to be substantially the same as the previous treatments.

As indicated above, it is important when heating tissue to be heat treated, that the surrounding normal tissue is not heated to an extent to damage the normal tissue. Therefore, it is important to monitor the temperature of at least the normal tissue at or near the outer edge of the tissue being heated. Systems such as shown in FIG. 1 include various temperature sensing apparatus and methods to monitor various tissue temperatures. In one embodiment of the prior art BSD-2000 system, it has been found that temperature of tissue being treated and surrounding tissue can be accurately monitored by a magnetic resonance imaging (MRI) system, and that the hyperthermia treatment can take place in the MRI system which then monitors the temperature of the tissue being treated and surrounding tissue during the treatment, see referenced U.S. Pat. No. 8,170,643. An embodiment of the BSD-2000 system for use with a prior art MRI system is shown in FIG. 2 where the MRI system is indicated generally by reference number 40, with the opening therein into which the patient is positioned is indicated by reference number 42. The various commercially available MRI systems generally have a support base extending from the MRI opening and a patient table structure that moves between the support base and the inside of the MRI opening to transport the patient, on a thin pad on the table, into the MRI system for diagnostic imaging. However, the normal MRI patient table structure is not configured to accept a hyperthermia support system. For a hyperthermia treatment in an MRI system, the patient, as positioned in applicator housing 14, has to be moved into and positioned in the MRI system. This requires a complete patient hyperthermia system that can be inserted into the MRI opening 42, such system including both the applicator housing 14 which applies the hyperthermia treatment and the patient support system (fabric sling 20) that supports, positions, and holds the patient in the appropriate position in the applicator housing 14. For an MRI hyperthermia system, a special narrow height system base 44, FIG. 2, which includes the applicator housing mounting track 32 and the vertical corner supports 28 for fabric sling 20, along with the hydraulic lift system for the vertical corner supports, has to be constructed along with a separate auxiliary supporting base 46 which supports the narrow height system base 44 at the appropriate height so it can be slide off the auxiliary supporting base 46 into the MRI opening 42. Further, since the various MRI system suppliers and the different MRI system models all have different support structures and patient transfer mechanisms, the hyperthermia systems for insertion into the MRI systems generally need small variations to fit different models of MRI systems. In addition, it has been found that even with the special narrow height system base 44, the patient and applicator array are at a very high position when that structure is placed on a standard support structure of an MRI system which often places the patient and reference devices used to compensate for magnetic field drift and non-uniformity out of the field of view of the MRI system.

There is a need for EMR applicator apparatus as part of a hyperthermia system that, together with a patient, can be placed on top of standard MRI system patient support surfaces so the patient in a hyperthermia applicator housing can be inserted into an MRI opening.

### SUMMARY OF THE INVENTION

According to the invention, it has been found that the EMR applicators of an EMR applicator array can be provided in an openable applicator housing to allow easy patient entrance to and exit therefrom so that the portion of the patient's body that contains the tissue to be treated can be positioned directly into the open applicator housing without the applicator housing extending completely around the patient's body. The applicator housing is then closed around the portion of the patient's body that contains the tissue to be treated so that the applicator housing then completely surrounds the portion of the patient's body containing the tissue to be treated. The closed applicator housing surrounding the patient's body does not need to be moved along the patient's body to the portion of the patient's body that contains the tissue to be treated. This can eliminate or reduce the need for full body supporting structure as part of the applicator housing. The applicator housing can provide support for the portion of the patient's body positioned therein, and support pads or pillows, separate from the applicator housing, can replace the need for a full body support, such as a full body length fabric sling of the prior art, as part of the applicator housing. Such an applicator housing can substantially reduce the size and complication of prior art applicator housings, and can provide an applicator housing that can be placed along with the patient, on a patient support surface, which can easily be inserted into a standard MRI system.

In one embodiment of the invention, the applicator housing includes a lower housing shell on a patient support surface and a separable upper housing shell. When the upper housing shell is aligned with and over the lower housing shell, a closed applicator housing is provided around the portion of the patient's body enclosed in the closed applicator housing and hyperthermia treatment can be provided to the patient. When the upper housing shell is removed to a rest position from above the lower housing shell, the lower housing shell is in an open condition on the patient supporting surface so a patient can position himself or herself in the open lower housing shell with the portion of the patient's body containing tissue to be treated directly above the lower housing shell. Foam padding or cushions on the patient support surface at both ends of the lower housing shell can support portions of the patient body extending from the ends of the lower support shell. With the patient positioned on the lower housing shell, the upper housing shell can be moved from its rest position to a treatment position over the lower applicator shell where the upper housing shell is held with respect to the lower housing shell so that the upper and lower housing shells together form a closed applicator housing around the portion of the patient's body containing the tissue needing hyperthermia treatment. Both the lower and upper applicator housing shell will include electromagnetic energy applicators, such as dipole antennas, arranged therein such that when in closed condition, at least one ring of electromagnetic energy applicators is provided around the portion of the patient's body enclosed in the applicator housing.

A special elongate patient support surface is preferably provided which can be easily slid into an MRI system and which includes rails for mounting the upper housing shell for movement along the patient support surface. This allows for movement along the rails of the upper housing shell from treatment position over the lower housing shell to a rest position away from the lower housing shell, usually toward an end of the patient support surface, to allow a patient to easily position himself or herself on the lower housing shell or to allow a patient to move from the lower housing shell. With the applicator housing shells separated, a patient can easily position himself or herself on the lower shell, such as by sitting on the patient support surface adjacent one end of the lower housing shell and then by lying down with the patient's posterior side below the portion of the patient's body having the tissue therein to be treated over the lower housing shell. When in position on the lower applicator shell, the upper housing shell can then easily be moved over the portion of the patient on the lower housing shell to form a completed housing surrounding the portion of the patient needing treating. The upper and lower housing shells will each contain a plurality of electromagnetic energy applicators arranged in an arc so that when the shells are positioned one above the other, at least one ring of applicators is provided surrounding the portion of the patient containing the tissue to be treated. Hyperthermia treatment can then be administered to the patient.

### THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of a free standing prior art BSD-2000 hyperthermia system;

FIG. 2 is a perspective view of a prior art modified BSD-2000 hyperthermia system adapted for use in combination with an MRI system;

FIG. 3 is a perspective view of a patient support surface configured to support a patient in a prone position and showing a lower housing shell adjustably positioned on the support surface for supporting the portion of the patient having the tissue needing treatment and showing cushions on either side of the lower housing and also showing a patient foot stop for positioning the patient. The upper housing usually positioned along the support surface is not shown;

FIG. 4 is a perspective view of the support surface shown in FIG. 3 and also showing the upper housing shell positioned in its normal rest position toward an end of the patent support surface thereby leaving the lower housing shell uncovered to provide easy access to the lower housing shell by the patient;

FIG. 5 is a perspective view of the support surface of FIGS. 3 and 4 showing the upper housing shell moved from its normal rest position shown in FIG. 3 to its treatment position over the lower housing shell;

FIG. 6 is perspective view of the patient support surface similar to that of FIG. 4 showing the upper housing shell in its rest position and showing a patient in position on the patient support surface and lower housing shell and showing the patient support surface positioned with respect to an MRI device so that the patient support surface can be slid into the MRI device;

FIG. 7 is a perspective view of the patient support surface of FIG. 5 with the upper housing shell moved into treatment position over the lower housing shell and over the portion of the patient with tissue therein needing treatment;

FIG. 8 is a perspective view of the lower housing shell alone;

FIG. 9 is a perspective view of the applicator housing formed when the upper housing shell in treatment position over the lower housing; and

FIG. 10 is a sectional view through the housing taken on the line 10-10 of FIG. 9.

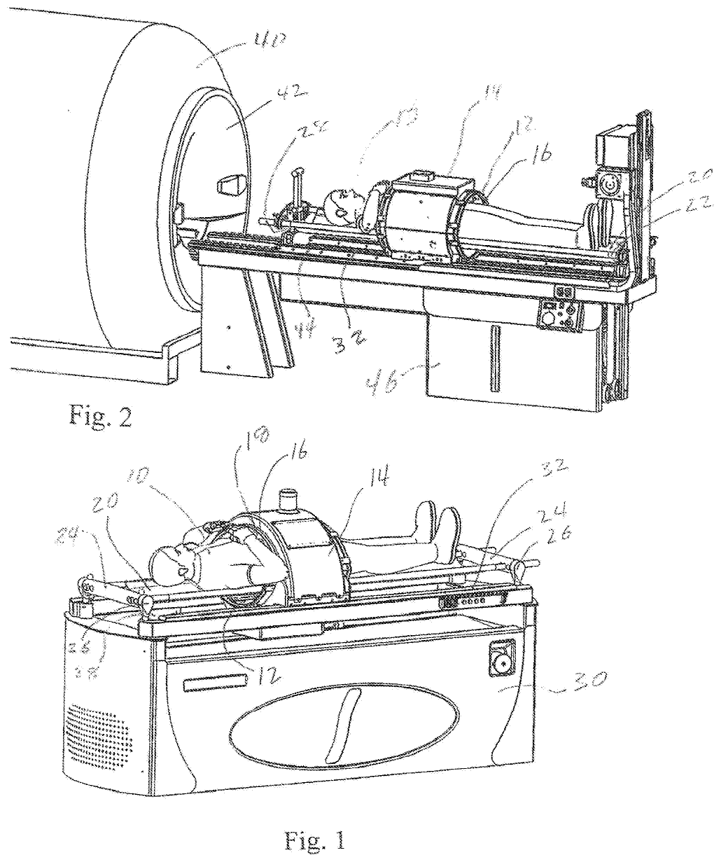

### DETAILED DESCRIPTION OF THE ILLUSTRATED EMBODIMENTS

The present invention provides an EMR applicator array housing that is openable to allow easy patient entrance to and exit therefrom so that the portion of the patient's body that contains the tissue to be treated can be easily positioned directly on the lower applicator housing shell of the applicator housing and the upper portion of the applicator housing shell can then be moved to position it over the lower applicator housing shell and the portion of the patient's body that contains the tissue to be treated. The lower applicator housing shell can provide support for the portion of the patient's body positioned therein, and support pads or pillows on a patient support surface, separate from the applicator housing, can support the portions of the patient's body extending from the ends of the lower applicator housing shell.

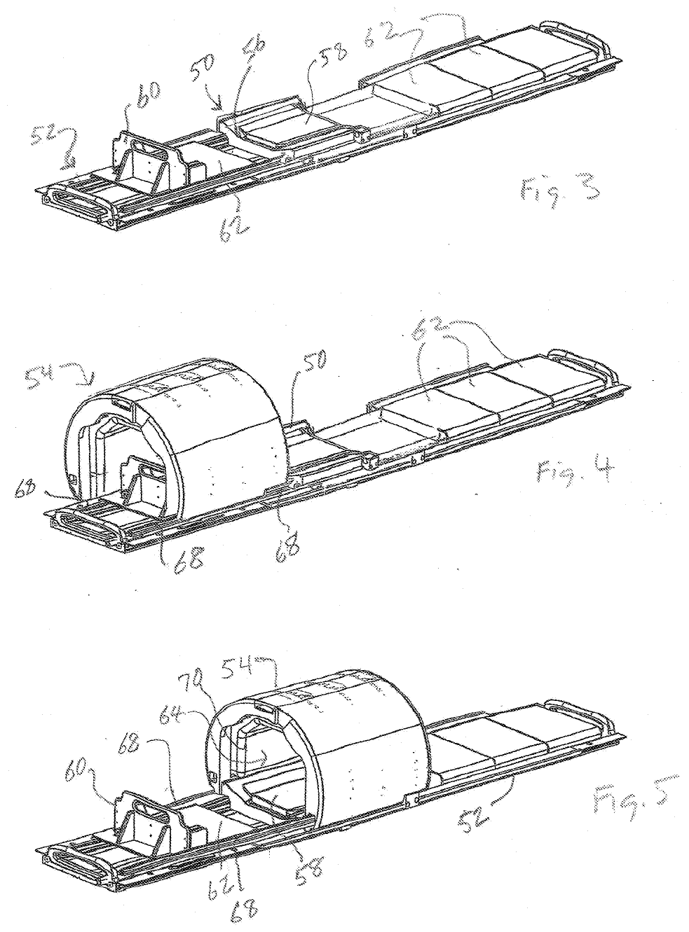

An example embodiment of the openable EMR applicator housing of the invention is illustrated as a lower applicator housing shell 50 positioned on a patient support surface 52 and a separable upper applicator housing shell 54. Such an applicator housing is shown in FIGS. 3-7. The lower applicator housing shell 50 is placed at a desired intermediate location along the patient support surface 52. Lower applicator housing shell 50 forms a lower, substantially concave surface 56 with a bolus 58 extending therefrom. It is advantageous to provide a foot stop 60 secured to the patient support surface 52 where a patent can place his or her feet when lying on the patient support surface. This foot stop 60 can be used to position the lower applicator housing shell. The position of the portion of the patient's body that requires hyperthermia treatment, i.e., the portion containing tissue or tumors needing treatment, can be measured for a standing patient from the floor on which a patient stands. The lower applicator housing shell is then positioned along the patient support surface at the approximate tissue or tumor location in the patient, which is the approximate distance measured from the foot stop 60 to a position within the lower applicator shell equal to the distance measured in the standing patient. With the lower applicator shell 50 positioned along the patient support surface 52, a patient is loaded into the applicator by sitting on the lower applicator shell. The patient then swivels to place his or her feet flat against the foot stop, lies down across the lower applicator shell, and straightens his or her legs to push his or her torso into position on the bolus of the lower applicator shell. The bolus 58 may be filled with a dielectric fluid, such as deionized water prior to receiving the patent thereon or can be filled with the dielectric fluid after the patient is received on the lower applicator housing shell. Cushions or pads 62 will be positioned on the patient support surface 52 at opposite sides of the lower applicator shell 50 to support the upper body and lower body portions of the patent extending beyond the ends of the lower applicator support 50. At this point, if desired, catheters with temperature sensors can be placed in natural body orifices of the patient and used during hyperthermia treatment for temperature measurement.

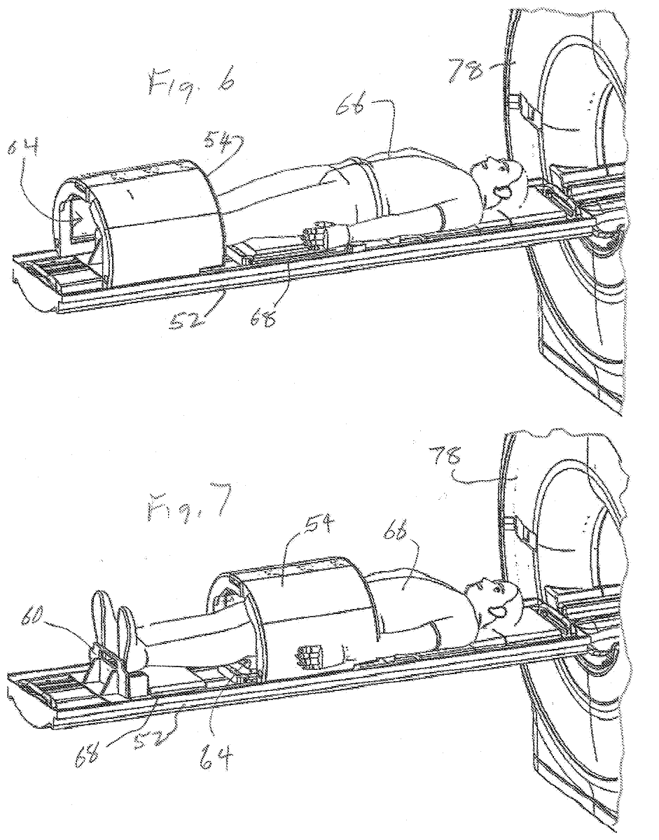

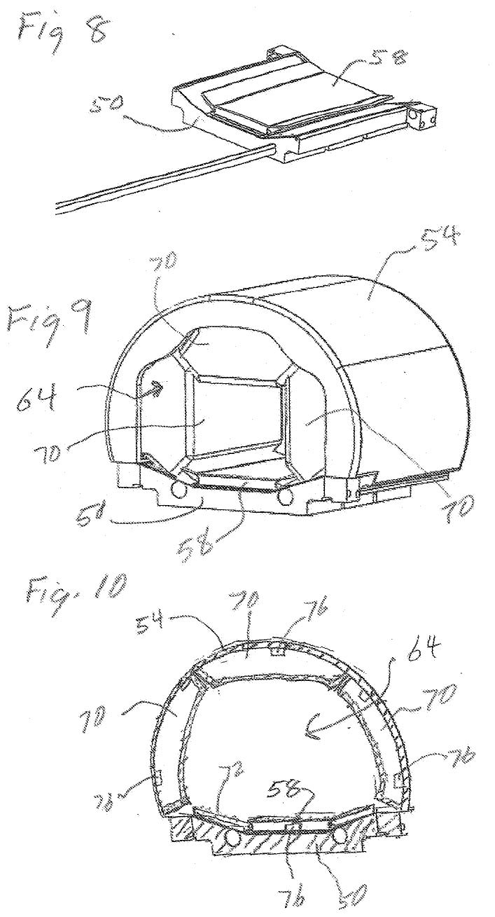

While not shown in FIG. 3 in order to more clearly show lower applicator housing shell 50 and foot stop 60 positioned on patient support surface 52 in FIG. 3, the applicator housing also includes separable upper housing shell 54 movable between a rest position shown in FIGS. 4 and 6 where upper applicator housing shell 54 is separated from lower applicator housing shell 50 so as to open the applicator housing and a closed position shown in FIGS. 5 and 7 where upper applicator housing shell 54 is over and aligned with lower applicator housing shell 50. When the upper applicator housing shell is removed to a rest position from above the lower housing shell, the lower housing shell is in an open condition on the patient supporting surface 52 so a patient 64, FIG. 6, can position himself or herself in the open lower applicator housing shell with the portion of the patient's body containing tissue to be treated directly above the lower applicator housing shell. When the upper applicator housing shell is in treatment position over the lower applicator housing shell, the upper and lower applicator housing shells enclose the portion of the patient with tissue needing hyperthermia treatment in an opening 64 between the upper and lower applicator housing shells.

In use, separable upper applicator housing shell 54 will be in rest position as shown in FIGS. 4 and 6 so a patient 66 can be easily positioned on lower applicator housing shell 50. Once the patient 66 is so positioned, upper applicator housing shell 54 is moved from rest position to treatment position shown in FIGS. 5 and 7 where it is aligned with and positioned over lower applicator housing shell 50 and over the portion of the patient 66 that is positioned over lower applicator housing shell 50, FIG. 7. For this purpose, rails 68 are provided along the opposite elongate top sides of patient support surface 52 with upper applicator housing shell 54 configured to rest on and move along such rails between its rest position and its treatment position over lower applicator housing shell 50. Rails 68 make it easy to move upper applicator housing shell 54 between the rest and treatment positions. When the upper housing shell 54 is aligned with and over the lower housing shell 50, a closed applicator housing is provided around the portion of the patient's body enclosed in the closed applicator housing. Means are provided to secure the upper housing shell in treatment position, if desired, such as a catch to hold the upper applicator housing shell in position on the rails 68. The upper housing shell can also be secured in rest position. The upper applicator housing shell also includes boluses 70 which are filled with dielectric fluid when the applicator housing is closed around the patient. Electromagnetic energy applicators, such as electromagnetic dipole antennas are arranged as applicators in the lower applicator housing shell and the upper applicator housing shell in such a manner that when the upper applicator housing shell is moved to closed position, at least one ring of electromagnetic applicators surround the opening 64 between the applicator housing shells and form at least one ring of applicators around the opening 64 and the patient's body portion in the opening. Hyperthermia treatment can then be provided to the patient.

FIG. 10 is a sectional view of the illustrated embodiment of the applicator housing of the invention. As shown in FIG. 10, lower applicator housing shell 50 forms an inner lower concave surface 72 and the upper applicator housing shell 54 forms an inner upper concave surface 74. The upper and lower applicator housing shells are made of a dielectric material such as a plastic material. When in the closed condition, the inner upper concave surface 74 faces the inner lower concave surface 72 to form the opening 64 between the upper and lower applicator housing shells through which the patient's body extends. EMR applicators, shown schematically as boxes 76, FIG. 10, and shown larger than actual scale size, are substantially evenly and/or symmetrically spaced and attached around the inside concave surfaces of the shells. Such EMR applicators can be in the form of dipole antennas formed on or attached to the inside concave surfaces of the upper and lower shells. Electrical cables, not shown, will connect the EMR applicators to a source of EMR energy outside of the applicator housing. Depending upon the hyperthermia system used, the EMR applicators can form a single ring of applicators around the shell or multiple rings of applicators around the shell. For example, if an applicator array configuration as shown in referenced U.S. Pat. No. 5,097,844 is to be provided to allow three dimensional positional steering and focusing of the heating pattern created by the applicator array, three rings of eight applicators each would be provided. Any desired number of rings and number of applicators per ring can be formed in the housing.

As indicated, when in closed condition, the inner upper concave surface 74 faces the inner lower concave surface 72 to form a cylindrical shell with an opening 64 extending from end to end therethrough. As used herein, cylindrical does not mean circular in cross section as the illustrated cylindrical shell and opening 64 extending therethrough is shown as being substantially elliptical in cross section, but merely means that it surrounds the patient. For treatment, the cylindrical shell is positioned around a portion of a patient's body, such as the patient's trunk or torso, containing the tissue to be treated. The upper portion and the lower portion of the patient's body extend from the ends of the applicator housing. As indicated, within the applicator housing, boluses 58 and 70 are provided which are filled with a dielectric fluid, such as deionized water, so that the bolus extends against the patient's body in opening 64 and provides a dielectric fluid in the space between the surface of the patient's body and the inside surface of the applicator housing.

The boluses are formed by flexible plastic material attached to the shells. The boluses can be inflated with a dielectric fluid, such as deionized water, to contact a portion of a patient body surface when the patient body portion is positioned in opening 64 created inside the housing when the housing is in closed condition as shown in FIGS. 5 and 7. The dielectric fluid in the boluses 58 and 70 will fill the area between the EMR applicators 76, FIG. 10, and the outside surface of the body received in the EMR applicator opening when closed, and the flexible material forming the boluses will conform to and abut the outside surface of the body to provide impedance matching between the EMR applicators and body. The bolus spaces will be connected to a source of dielectric fluid so they can be controllably filled and emptied when desired and to the extent desired. The boluses may all be connected together and to the source of dielectric fluid so are all inflated with dielectric fluid at the same time and to the same pressure, or the bolus in the lower applicator housing shell may be connected separately to a source of dielectric fluid so can be inflated at a different time and to a different pressure than the boluses in the upper applicator housing shell. In this way the bolus in the lower applicator housing shell can be inflated before the patient is positioned on the lower applicator housing shell so is supported by the bolus when being positioned on the lower applicator shell similarly to a water bed.

As shown in FIGS. 6 and 7, the patient support surface 52 will be similar to the standard patient support surfaces used with MRI devices and although no support is shown for the patient support surface 52 in the current drawings, the patient support surface will be supported at the entrance to an MRI device 78, FIGS. 6 and 7, in the standard manner as currently used for MRI devices. Such patient support surface with the patient thereon will be inserted into the MRI device so the hyperthermia treatment is performed in the MRI Device where the MRI system is used to monitor temperature of the tissue within the body being treated and the tissue surrounding the tissue being treated.

Whereas the invention is here illustrated and described with reference to an embodiment thereof presently contemplated as the best mode of carrying out the invention in actual practice, it is to be understood that various changes may be made in adapting the invention to different embodiments without departing from the broader inventive concepts disclosed herein and comprehended by the claims that follow:

1. An electromagnetic energy applicator housing for positioning an array of electromagnetic energy applicators around an opening adapted to receive a portion of a patient body having tissue therein in need of hyperthermia treatment, wherein the electromagnetic energy applicator housing can move between an open condition to directly receive a portion of the patient body having the tissue therein in need of hyperthermia treatment and a closed condition for treatment, comprising:
an elongate patient support surface for supporting the electromagnetic energy applicator housing and a patient to receive hypothermia treatment positioned in the electromagnetic energy applicator;
a lower applicator housing shell adjustably mounted on the patient support surface at a desired position along the elongate patient support surface, said lower applicator housing shell forming an inner lower substantially concave surface;
a lower bolus extending from the inner lower concave surface of the lower housing shell and adapted to be filled with a dielectric fluid, said lower bolus having a lower bolus surface spaced from the inner lower concave surface when filled with a dielectric fluid and adapted to receive a portion of the patient body having tissue therein in need of hyperthermia treatment when the portion of the patient body having tissue therein in need of hyperthermia treatment is to be received in the electromagnetic energy applicator housing;
an upper applicator housing shell movably mounted on the patient support surface to be moved from a rest position along the elongate patient support surface to a treatment position over the lower applicator housing shell, said upper applicator housing shell forming an inner upper concave surface that faces the inner lower concave surface to create an opening between the upper housing shell and the lower housing shell when the upper applicator housing shell is in treatment position over the lower applicator housing shell;
an upper bolus extending from the inner upper concave surface of the upper applicator housing shell and adapted to be filled with a dielectric fluid;
a plurality of electromagnetic energy applicators positioned on the inner lower concave surface of the lower housing shell and the inner upper concave surface of the upper housing shell so as to create, when the upper housing shell is in treatment position over the lower housing shell, at least one ring of a plurality of electromagnetic energy applicators around the opening between the lower housing shell and the upper housing shell adapted to receive the portion of the patient body therein for hyperthermia treatment.

2. The electromagnetic energy applicator housing according to claim 1, wherein the elongate patient support surface includes opposite elongate top sides and additionally including rails along the upper opposite elongate top sides of patient support surface and the upper applicator housing shell is configured to rest on and move along such rails between its rest position and its treatment position.

3. The electromagnetic energy applicator housing according to claim 2, additionally including securing means to secured the upper applicator housing shell in treatment position when moved into treatment position.

4. The electromagnetic energy applicator housing according to claim 3, additionally including securing means to secured the upper applicator housing shell in rest position when moved into rest position.

5. The electromagnetic energy applicator housing according to claim 1, additionally including securing means to secured the upper applicator housing shell in treatment position when moved into treatment position.

6. The electromagnetic energy applicator housing according to claim 5, additionally including securing means to secured the upper applicator housing shell in rest position when moved into rest position.
